(19) United States
(12) Patent Application Publication
(10) Pub. No.: US 2006/0159658 A1
(43) Pub. Date: Jul. 20, 2006

(54) Methods of treating disease with glycosylated interferon

(75) Inventors: Yashwant M. Deo, East Brunswick, NJ (US); Stephen H. Parker, Jefferson, GA (US); Markley C. Leavitt, Watkinsville, GA (US)

Correspondence Address: AVIGENICS, INC., 111 RIVERBEND ROAD, ATHENS, GA, 30605, US

(73) Assignee: AviGenics, Inc.

(21) Appl. No.: 11/335,375

(22) Filed: Jan. 19, 2006

Related U.S. Application Data
(60) Provisional application No. 60/645,059, filed on Jan. 19, 2005.

(51) Int. Cl. A61K 38/21

(52) U.S. Cl. 424/85.7

(57) ABSTRACT

This invention includes methods for treating conditions with pharmaceutical compositions that comprise glycosylated interferon alpha. Pharmaceutical compositions that can be employed in the present invention include glycosylated interferon alpha 2 species in combination with pharmaceutical carriers, pharmaceutical excipients and/or other agents.

### RELATED APPLICATION INFORMATION

This application claims the benefit of U.S. Provisional Patent Application No. 60/645,059, filed Jan. 19, 2005, the disclosure of which is incorporated in its entirety herein by reference.

### BACKGROUND OF THE INVENTION

The human body makes endogenous interferon as part of the immune response, particularly when the body reacts to cancer or infection. Interferon stimulates the immune system by activating killer T-cells and other cells that normally attack cancer cells and by encouraging cancer cells to release chemicals that attract the cells of the immune system. Interferons can be used as drugs in monotherapy or in combination therapy with other drugs (including protein drugs such as cytokines and hormones) surgery, chemotherapy and/or radiation treatment.

Interferons are classified as alpha, beta, and gamma, and are designated according to their ability to stimulate an immune response (antigenic type). Alpha interferons are produced by leukocytes or lymphoblastoid cells. Certain human genes encode subspecies of interferon alpha, which can be further separated into classes 1 and 2. Beta interferons are produced by skin fibroblasts (connective tissue cells), and gamma interferons are produced by T lymphocytes and are sometimes called “immune” interferons (see Christine Verini, U.S. Pharmacist 23:5, a Jobson Publication).

Interferon can be administered as a drug to patients and is used to treat several different types of cancer, including malignant melanoma, multiple myeloma, some types of leukemia, eye cancer, kidney cancer, liver cancer, bladder cancer and others. For example, kidney cancer can be treated with interferon alpha 2a (e.g., Roferon®-A interferon by Hoffmann-La Roche). Malignant melanoma can be treated with interferon alpha 2b (e.g., Intron®-A interferon by Schering-Plough). Interferon is known to be administered intravenously (IV), by intramuscular injection (IM), by subcutaneous injection (SQ) under the skin, or on the skin.

Interferon is also used to treat viral infections. Interferon is known to be secreted by cells in response to viral infections. Particularly, interferons are released by virus-infected cells, wherein they assist normal cells to make antiviral proteins. As a result, there are various interferons that are administered to patients by intramuscular or subcutaneous injection for the treatment of chronic hepatitis B and chronic hepatitis C. Interferons exert their effects by binding to specific membrane receptors. This receptor binding initiates a series of intracellular signaling events that ultimately leads to enhanced expression of certain genes. This in turn leads to the enhancement and induction of certain cellular activities including augmentation of target cell killing by lymphocytes and inhibition of virus replication in infected cells. Various recombinant forms of interferon alpha (interferon alpha 2a and interferon alpha 2b) and a recombinant non-naturally occurring type I interferon (interferon alfacon-1) are approved for treatment of viral hepatitis. In certain instances, combination therapy using interferon and ribavirin has proven effective treatment against hepatitis C. See, for example, U.S. Pat. No. 6,472,373, issued Oct. 29, 2002 and U.S. Pat. No. 6,299,872, issued Oct. 9, 2001, the disclosures of which are incorporated in their entirety herein by reference.

Non-glycosylated interferon alpha 2b (e.g., Intron®-A interferon by Schering-Plough) is effective in the treatment of adults with chronic hepatitis B virus infection and evidence of viral replication. When a patient is infected with hepatitis B virus (HBV) it is indicated by the presence of hepatitis B surface antigen in the blood. The patient often has an ongoing inflammation of the liver, evidenced by an elevation in serum aminotransferase activities. A recommended dose of Non-glycosylated interferon alpha 2b for the treatment of chronic hepatitis B is, for example, 5,000,000 units daily, administered by subcutaneous or intramuscular injection for 16 weeks. The patient is typically monitored during the treatment period for adverse side effects including flu-like symptoms, depression, rashes, chills, shakes, muscle aches, other reactions and abnormal blood counts.

Wellferon® (GlaxoWellcome) is obtained from cell lines in which interferon production has been induced and includes a mixture of glycosylated interferons. Certain inherent limitations exist relating to the manufacture and use of Wellferon®. For example, large scale production of Wellferon® may be prohibitive. In addition, obtaining formulation consistency may be difficult or impossible due to the complicated mixture of interferons present in Wellferon®.

Interferon alpha 2a (e.g., Roferon®-A interferon by Hoffmann-La Roche), interferon alpha 2b (e.g., Intron®-A interferon by Schering-Plough) and interferon alfacon-1 (e.g., Infergen® interferon by Amgen) are all approved in the United States for the treatment of adults with chronic hepatitis C. The recommended dose of interferons alpha 2b and alpha 2a for the treatment of chronic hepatitis C is, for example, 3,000,000 units three times a week, administered by subcutaneous or intramuscular injection. Although, six months of treatment was originally recommended for interferons alpha 2a and alpha 2b, several studies have shown that treatment for a year or longer may be preferable (Poynard et al. (1996) Hepatology 24:778-789). In fact, treatment of patients for 1 to 2 years with interferons alpha 2a and alpha 2b is now approved by the FDA. The results of several published clinical studies demonstrate that about 50 to 70 percent of patients with chronic hepatitis C can respond to treatment with interferon alpha 2b which can be demonstrated by reductions in the serum aminotransferase activities to near normal. However, frequent administration over extended periods of time is typically required.

There remains a need for improved methods of treating disease, for example, with agents having improved pharmacokinetic characteristics, a greater treatment success rate, improved formulation consistency, fewer side effects and having a broad spectrum of use.

### SUMMARY OF THE INVENTION

The present invention relates to the discovery that pharmaceutical compositions comprising glycosylated alpha interferons, for example, interferon alpha 2 (e.g., interferon alpha 2b) can be used to more effectively treat conditions such as cancerous conditions and/or viral conditions in a subject relative to non-glycosylated alpha interferons or mixed interferons. For example, the invention contemplates the use of purified glycosylated interferon alpha (for example, interferon alpha 2, e.g., interferon alpha 2b) in a suitable pharmaceutical composition. The glycosylated alpha interferons employed in the invention appear to stimulate the immune system to a greater degree than non-glycosylated alpha interferons. One advantage of the present invention is that a subject may be administered pharmaceutical compositions of glycosylated interferon alpha, in relatively lower doses thus achieving substantially similar or greater benefits than obtained with a higher dose of a conventional or standard recombinant interferon. Another advantage is that a subject may be administered pharmaceutical compositions of glycosylated interferon alpha, for a relatively shorter period of time to achieve substantially the same or greater benefits than is the case with conventional standard recombinant interferons administered for a greater period of time. In addition, the methods may have fewer unwanted side effects than conventional treatments with standard recombinant interferons. In addition, in certain instances, the methods can provide for a decreased antigenicity relative to methods involving administration of non-glycosylated intereferon alpha.

Glycosylated interferon alpha 2b has previously been produced in avians for research purposes. Such research provided valuable information including evidence that avians such as chickens could withstand substantial quantities of substances such as interferon in the blood stream with no apparent toxic effect. In addition, among other things, it was shown that human interferon and other proteins produced under the control of a CMV promoter would be produced in oviduct tissue at surprisingly high levels. Furthermore, it was shown that the glycosyaltion pattern of human proteins produced in chickens can be substantially similar to the natural glycosylation pattern of the same protein produced in humans.

Surprisingly, glycosylated interferon alpha such as glycosylated interferon alpha 2b produced in avians, is shown to have significantly enhanced therapeutic properties relative to substantially the same alpha interferon but in non-glycosylated form.

The present invention provides for administration of glycosylated interferon alpha to a subject through various different routes depending on the condition of the subject to be treated. For example, a condition such as cancer or a viral infection may be treated by systemically administering pharmaceutical compositions of glycosylated interferon alpha to a patient, for example, through controlled release capsules, implants or injections. Similarly, pharmaceutical compositions of glycosylated interferon alpha may be administered locally such as orally, nasally or through injections. As such, the present invention allows for certain treatments of conditions (e.g., diseases) without being limited to specific routes of administration.

Pharmaceutical compositions of the present invention may include glycosylated interferon alpha species or combinations thereof in combination with pharmaceutical carriers, pharmaceutical excipients and/or other agents. The glycosylated interferon alpha includes, but is not limited to, interferon alpha, such as interferon alpha 2 (e.g., interferon alpha 2b) glycosylated at position 106 (e.g., Thr-106) such as human glycosylated interferon alpha and poultry-derived glycosylated interferon alpha. The pharmaceutical compositions may include glycosylated interferon alpha species or combinations thereof in combination with pharmaceutical carriers, pharmaceutical excipients and/or other agents.

One aspect of the present invention provides for a method for treating a condition in a subject including administering to the subject a therapeutically effective amount of a pharmaceutical composition comprising glycosylated interferon alpha. The method may include monitoring the subject to detect any amelioration of the condition. In one embodiment, the cancerous condition may not be substantially ameliorated by administering a pharmaceutical composition comprising the same interferon alpha that is in non-glycosylated form. In another embodiment, the cancerous condition may be less ameliorated by administering a pharmaceutical composition comprising an interferon alpha that is non-glycosylated relative to the administration of a substantially similar or the same interferon alpha that is glycosylated.

Conditions contemplated for treatment as disclosed herein include any condition which may be ameliorated by administering glycosylated interferon alpha including cancer, viral conditions and other conditions such as multiple sclerosis, arthritis, atherosclerosis, fibromyalgia syndrome, chronic fatigue syndrome, pneumonia, and endothelial dysfunctions.

The present invention provides treatment for various cancerous conditions including, but not limited to, skin cancer (e.g., melanoma), leukemia (e.g., hairy cell leukemia, chronic myeloid leukemia), kidney cancer, liver cancer, bladder cancer, lymphoma and Kaposi's sarcoma.

The present invention contemplates treatment for various viral conditions including, but not limited to, hepatitis including hepatitis B and hepatitis C, venereal warts, cardiomyopathy, Epstein-Barr infection, chronic fatigue, HIV and measles. In one embodiment, the viral condition is a chronic condition, for example, chronic hepatitis C. In certain embodiments, the methods of the present invention are employed to treat a subject that is a patient infected with hepatitis C virus (HCV) or a patient infected with HCV and further infected with human immunodeficiency virus (HIV). In order to treat a subject for a viral condition, different routes of administration may be selected. For treatment of a viral condition, the interferon alpha may be administered in combination with at least one additional agent. The agents may be administered simultaneously or sequentially. Such agents include, but are not limited to, viramidine and ribavirin.

Another aspect of the present invention provides methods for treating a viral condition in a subject including administering to the subject a therapeutically effective amount of a pharmaceutical composition comprising glycosylated interferon alpha in combination with ribavirin or viramidine. Other agents, pharmaceutical carriers and/or excipients may further be included in the pharmaceutical compositions.

In one embodiment, the methods of the present invention encompass treating a condition in a subject, such as a cancerous or viral condition, by administering to the subject a therapeutically effective amount of a pharmaceutical composition with glycosylated interferon alpha 2. In one embodiment, the glycosylated interferon alpha 2 has the amino acid sequence of human interferon alpha 2a or has the amino acid sequence of human interferon alpha 2b. In one embodiment, the glycosylation pattern of the interferon is similar to or the same as the human interferon. In one particularly useful embodiment, the glycosylated interferon alpha 2 is poultry-derived glycosylated human interferon alpha 2, as disclosed in, for example, U.S. Pat. No. 6,730,822, issued May 4, 2004, the disclosure of which is incorporated in its entirety herein by reference. Use of interferon alpha 2, for example, interferon alpha 2b, produced in poultry can be particularly advantageous, for example, from the standpoint of a low cost of production and the similarity of glycosylation patterns of native human interferons compared to the human interferons produced in poultry. Also, therapeutic proteins such as interferon produced in the avian oviduct and packaged in eggs can be purified very efficiently compared to production of similar proteins in cell lines.

Glycosylated interferon alpha 2 contemplated for use as disclosed herein includes, but is not limited to, glycosylated interferon alpha 2a, glycosylated interferon alpha 2b and glycosylated interferon alpha 2c. In certain embodiments, though treatment of the condition is ameliorated by administering the glycosylated interferon alpha, the condition may not be substantially ameliorated by administering a pharmaceutical composition comprising an otherwise similar or the same (i.e., having the same amino acid sequence) interferon alpha that is in non-glycosylated form.

The invention also relates to stimulating an immune response by administering glycosylated interferon alpha, for example interferon alpha 2 (e.g., interferon alpha 2b). Stimulation of the immune response can include, without limitation, stimulation of T cells, stimulation of B cells and stimulation of antibody production.

Where the present application discloses uses or methods of treatment using glycosylated interferon alpha, it is also contemplated that each such use or method of treating relates to glycosylated forms of each subtype of interferon alpha (e.g., interferon alpha 1 and interferon alpha 2) and relates to glycosylated forms of each sub-subtype of alpha interferon (e.g., interferon alpha 2a, interferon alpha 2b and interferon alpha 2c).

These and other objects, advantages and embodiments of the present invention will be apparent when read with the detailed description and claims which follow.

Any combination of features described herein is included within the scope of the present invention provided that the features included in any such combination are not mutually inconsistent. Such combinations will be apparent based on this specification and on the knowledge of one of ordinary skill in the art.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds.

Definitions and Abbreviations

The following definitions and abbreviations are set forth to illustrate and define the meaning and scope of the certain terms used to describe the present invention.

The term “condition” refers to a defective state of health, for instance, a disease.

The term “cancerous condition” refers to a malignant neoplastic condition, which is any malignant growth or tumor caused by abnormal and uncontrolled cell division; it may spread to other parts of the body through the lymphatic system or the blood stream. A cancerous condition, as referred to herein, also includes a premalignant condition. A premalignant condition exists when any tissue that is not yet malignant, is poised to become malignant. Appropriate clinical and laboratory tests are designed to detect premalignant tissue while it is still in premalignant stages. Treatment is used to eliminate or kill the premalignant tissue, thereby preventing the development of cancer. The proper treatment method depends on the particular premalignant tissue involved and can be determined based the specification in combination with the knowledge of a clinician of ordinary skill in the field.

The term “derived” means obtained from.

The term “IU” means international unit. In one embodiment, the value of an IU is defined by the World Health Organization. Other certain standardized measurements for IU are contemplated for use herein. See, for example, Meager, et al. (2001) J. Immunological Methods, 257, 17-33, the disclosure of which is incorporated in its entirety herein by reference.

The abbreviation “mg” means milligram and the abbreviation “ml” means milliliter.

The term “viral condition” refers to an infection or inflammation caused by viruses. Viruses can cause relatively harmless conditions, for example, warts or can cause devastating conditions such as AIDS. Examples of viral conditions contemplated for treatment in accordance with the present invention include hepatitis B, hepatitis C, warts and measles. Measles is an acute and highly contagious viral disease marked by distinct red spots followed by a rash. Measles occurs primarily in children but can also occur in adults. Warts are caused by the Human papilloma virus (HPV) and may be transmitted sexually (i.e., venereal warts). Papilloma viruses cause small growths (warts) on the skin and mucous membranes.

“Hepatitis C” is an inflammation of the liver caused by the hepatitis C virus (HCV). About 50 percent of people infected with hepatitis C will develop chronic hepatitis which is a continuing inflammation of the liver that damages the liver cells. “Hepatitis B” is an inflammation of the liver caused by the hepatitis B virus (HBV). Once infected with the hepatitis B virus, approximately 10 percent of the people develop a chronic permanent infection (chronic carrier state) of which some will develop slow but progressive liver damage leading to cirrhosis or hepatocellular cancer. Hepatitis B is a major cause of liver cancer.

A “subject” is an animal patient including a human patient.

The term “glycosylated” as in “glycosylated interferon alpha”, refers to a protein such as interferon alpha that has been modified by the attachment of carbohydrates (sugar molecules) to one or more amino acids of the protein. Many naturally occurring proteins are glycosylated, for example, interferon alpha can be naturally glycosylated. Thus, a glycoprotein is a macromolecule composed of a protein and carbohydrate(s). The carbohydrate can be attached to the protein by a posttranslational modification, at either, for example, asparagine, hydroxylysine, hydroxyproline, serine, or threonine. Possible carbohydrates include, but are not limited to, glucose, glucosamine, galactose, galactosamine, mannose, fructose, and sialic acid. The sugar group can assist in protein folding and can be important for stability of the protein. Glycoproteins are also important for immune cell recognition, especially in mammals.

“Ameliorate”, as referred to herein, means any improvement of a condition. An improvement of a condition may be detected after treatment of the condition has been initiated.

### DETAILED DESCRIPTION OF THE INVENTION

The present invention provides for methods of treating conditions in a subject including administering to the subject a therapeutically effective amount of glycosylated interferon alpha. The methods may include monitoring the subject to detect any amelioration of the condition. In one embodiment, the glycosylated interferon alpha is in a pharmaceutical composition. The pharmaceutical composition can include a pharmaceutical carrier and/or other agents. In one embodiment, the pharmaceutical composition excludes or substantially excludes interferon other than glycosylated interferon alpha. In another useful embodiment, the pharmaceutical composition excludes or substantially excludes any interferon other than glycosylated interferon alpha 2. In another useful embodiment, the pharmaceutical composition excludes or substantially excludes any interferon other than glycosylated interferon alpha 2b.

In one embodiment, compared to treatment of a condition resulting in substantial amelioration of the condition by administering a glycosylated interferon alpha as disclosed herein, the condition is not substantially ameliorated by administering a pharmaceutical composition comprising an interferon alpha with the same or substantially the same amino acid sequence, but non-glycosylated. For example, the condition may only improve by a limited amount upon administration of a non-glycosylated alpha interferon and not improve further, or improve only slightly, with continued administration of the non-glycosylated alpha interferon. In another example, the condition may improve initially upon administration of a non-glycosylated alpha interferon and then decline in spite of continued treatment with the non-glycosylated alpha interferon.

The methods of this invention encompass the use of glycosylated interferon alpha 2b for treatment of viral and cancerous conditions of mammalian subjects including human patients. The invention contemplates the administration of any useful glycosylated interferon alpha, for example, the administration of any useful glycosylated interferon alpha 2 such as glycosylated interferon alpha 2b. Any useful interferon glycosylation pattern is contemplated for use herein. For example, the interferon can be glycosylated by CHO cells, human cells (e.g., produced in a human cell line), mouse cells (e.g., produced in a mouse cell line) or any other useful cells. In addition, the glycosylated interferons contemplated for use in the present invention can be glycosylated in a transgenic animal such as a transgenic cow, a transgenic mouse or a transgenic goat. In one useful embodiment, the interferon is glycosylated in a transgenic avian (e.g., a transgenic chicken). The invention also contemplates use of the interferons wherein the interferons have been glycosylated in vitro by methods of chemical synthesis as is understood by practitioners of skill in the art.

The nucleotide coding sequences and corresponding amino acid sequences for many interferon alphas are well known in the art. For example, the coding sequence and amino acid sequence for natural human IFN alpha 2b includes 498 nucleotides (NCBI Accession Number AF405539 and GI:15487989) and 165 amino acids (NCBI Accession Number AAL01040 and GI:15487990).

The structure of human glycosylated interferon alpha 2 differs from that of its recombinant Escherichia coli-derived equivalent, i.e., natural interferon alpha 2 is significantly more hydrophilic and the molecular mass of the natural protein is higher than that of recombinant interferon alpha 2. Natural human interferon alpha 2 contains the disaccharide galactosyl-N-acetylgalactosamine (Gal-GalNAc) linked to Threonine-106 (Thr-106). In certain of the molecules, this core carbohydrate carries (alpha-)N-acetylneuraminic acid, whereas a disaccharide, likely N-acetyl-lactosamine, is bound to Gal-GalNAc in molecules. Additional glycosylation isomers are also present in small amounts.

Interferon alpha 2 may be the only known interferon alpha species with a threonine residue at position 106 (see Adolf et al. (1991) Biochem. J. 276:511-518). Further, two out of nine of the interferon subtypes produced by leukocytes after a Sendai-virus induction are found to be glycosylated, namely interferon alpha 14c and interferon alpha 2b (see Nyman et al. (1998) Eur. J. Biochem. 253:485-493). Interferon alpha 14 is the only interferon alpha subtype with potential N-glycosylation sites, asparagines 2 and 72 (Asn-2 and Asn-72, respectively), but only Asn-72 is actually glycosylated. These interferon alphas are illustrative examples of interferons which are contemplated for use as disclosed herein.

The oligosaccharide chains of naturally occurring interferons have been isolated and analyzed by mass spectrometry and specific glycosidase digestions (see Nyman et al., supra). Both interferon alpha 2b and interferon alpha 14c resolve into three peaks in reversed-phase high performance liquid chromatography (RP-HPLC). Electrospray ionization mass spectrometry (ESI-MS) analysis of interferon alpha 2b fractions from RP-HPLC reveal differences in their molecular masses, suggesting that these represent different glycoforms. This is confirmed by mass-spectrometric analysis of the liberated O-glycans of each fraction. Interferon alpha 2b is estimated to contain about 20 percent of the core type-2 pentasaccharide, and about 50 percent of di-sialylated and 30 percent of mono-sialylated core type-1 glycans (see Nyman et al. and Adolf et al., supra). The glycosylation can have positive effects including, but not limited to, enhanced pharmacokinetics, pharmacodynamics and stability. Such characteristics can be particularly important when interferon is administered to patients in the form of drugs.

The present invention encompasses the use of a novel transgenic poultry-derived glycosylated human interferon alpha 2b (TPD IFN alpha 2b) derived from avians. See, for example, US Patent application publication No. 20040019923, published Jan. 29, 2004, the disclosure of which is incorporated herein in its entirety by reference which discloses the production of, and characterizes, transgenic poultry-derived glycosylated human interferon alpha 2b (TPD IFN alpha 2b). TPD IFN alpha 2b exhibits a novel glycosylation pattern and contains two new glycoforms. TPD IFN alpha 2b O-linked carbohydrate structures have certain similarities to human interferon alpha 2b. Therefore, the avian glycosylated form of the interferon alpha, such as interferon alpha 2b, may be substantially superior to other glycosylation forms (e.g., CHO cell glycosylation pattern) of the same interferon.

A carbohydrate analysis of TPD interferon, including a monosaccharide analysis and FACE analysis, reveals the sugar make-up or novel glycosylation pattern of the protein. As such, TPD IFN alpha 2b shows the following monosaccharide residues: N-Acetyl-Galactosamine (NAcGal), Galactose (Gal), N-Acetyl-Glucosamine (NAcGlu), and Sialic acid (SA). TPD IFN alpha 2b is O-glycosylated at Thr-106. This type of glycosylation is similar to human interferon alpha 2, wherein the Thr residue at position 106 is unique to interferon alpha 2. Similar to natural interferon alpha, TPD IFN alpha 2b does not have mannose residues.

In one useful embodiment of the invention, glycosylated interferon alpha 2b is shown to be superior to non-glycosylated interferon alpha 2b. For example, glycosylated interferon alpha 2b has been shown to be an improved drug relative to non-glycosylated interferon alpha 2b as determined by neopterin analysis, beta-2-microglobulin analysis, 2′5′ OAS (oligoadenylate synthetase) analysis and protein kinase RNA analysis (each analysis performed by methods well know in the art). In addition, glycosylated interferon alpha 2b may show an unexpectedly high biological activity in vivo and/or can be well tolerated (e.g., no adverse side effects) in vivo at doses expected to be therapeutically effective.

The glycosylated interferon alpha, for example, glycosylated interferon alpha 2 (e.g, glycosylated interferon alpha 2b), is to be administered in a therapeutically effective amount during the treatment of conditions in accordance with the present invention. A therapeutically effective amount can be determined by a skilled practitioner such as a physician of ordinary skill in the art. A therapeutically effective amount of the glycosylated interferon alpha, for example, glycosylated interferon alpha 2 (e.g, glycosylated interferon alpha 2b), can be in the range of from about 0.1 to about 100 million IU, in single or divided doses. Administration of the doses is typically optimized as determined by certain factors including the condition treated and its severity. In one embodiment, the doses are administered at a frequency in a range of between once per day and once every six months.

In one embodiment, the dose may be administered one time per week or more often (e.g., 2 times per week, or 3 times per week, or 4 times per week, or 5 times per week, or 6 times per week, or 7 times per week, for example, once per day) or less often (e.g., once every 2 weeks, or once per month, or once per 2 months, or once per 3 months, or once per 4 months, or once per 5 months, or once per 6 months).

Glycosylated interferon alpha may be administered as single or divided doses. In one embodiment, the therapeutically effective amount of glycosylated interferon administered during the treatment of conditions in accordance with the present invention is in the range of from about 1 to about 200 million IU once per week or more often or less often. In one embodiment, the therapeutically effective amount of glycosylated interferon administered during the treatment of conditions in accordance with the present invention is in the range of from about 1 to about 100 million IU once per week or more often or less often. In another embodiment, the therapeutically effective amount of glycosylated interferon administered during the treatment of conditions in accordance with the present invention is in the range of from about 1 to about 50 million IU once per week or more often or less often. In another embodiment, the glycosylated interferon can be administered in the range of from about 0.1 to about 20.0 micrograms per kilogram once per week, or in the range of from about 0.1 to about 10.0 micrograms per kilogram twice per week. In another embodiment, the glycosylated interferon can be administered in the range of from about 0.01 to about 5.0 micrograms per kilogram once per week, or in the range of from about 0.01 to about 5.0 micrograms per kilogram twice per week. In another embodiment, the glycosylated interferon can be administered in the range of from about 0.70 to about 1.6 micrograms per kilogram or about 0.35 to about 0.8 micrograms per kilogram twice per week. The therapeutically effective amount of glycosylated interferon alpha 2b is administered to the patient is typically in the form of a pharmaceutical composition. The above given dosage regimes are examples and should not be construed to limit the invention. As will be apparent to one of skill in the art, additional regimes are also encompassed by the present invention.

The invention also contemplates the administration of one or more additional agents before, after or concurrently with the glycosylated interferon alpha. For example, additional agents such as ribavirin or viramidine can be administered to the patient having chronic hepatitis C in association with glycosylated interferon alpha, i.e., before, after or concurrently of glycosylated interferon alpha. In one embodiment, a pharmaceutical composition is administered which includes the glycosylated interferon alpha and the one or more agents.

In one embodiment, the dosage of glycosylated interferon alpha is administered during the same period of time that the patient receives specific doses of ribavirin or viramidine. The amount of ribavirin or viramidine administered concurrently with the glycosylated interferon alpha may be, for example, and without limitation, from about 50 to about 4000 mg per day, or from about 100 to about 1700 mg per day, or about 400 to about 1300 mg per day, or about 500 to about 900 mg per day or about 300 to about 700 mg per day.

A specific condition may be treated by systemically administering pharmaceutical compositions of glycosylated interferon alpha to a patient, for example, through controlled release capsules, implants or injections. Similarly, pharmaceutical compositions of glycosylated interferon alpha may be administered locally such as orally, nasally (e.g., aerosol nasal spray) or through injections. More specifically, the methods of administering the glycosylated interferon alpha in accordance with the present invention include, but are not limited to, parenteral administration such as by subcutaneous, intravenous, or intramuscular injection as well as oral administration such as via capsules or tablets. Other agents such as ribavirin or viramidine can be administered orally in capsule, tablet, or liquid form, intranasally as an aerosol by nasal spray, or parenterally, by subcutaneous, intravenous, or intramuscular injection. Ribavirin or viramidine can be orally administered in association with the parenteral administration of glycosylated interferon alpha.

As the skilled artisan will appreciate, other types of administration of medications of the invention, as they become available, are encompassed, such as transdermally, by suppository, by various sustained release forms, and by pulmonary inhalation. Any administration that assures that the proper dosages are delivered without affecting the active ingredient is contemplated by the present invention.

Solid form preparations of glycosylated interferon alpha include powders, tablets, dispersible granules, capsules, cachets and suppositories. In one embodiment, powders and tablets are comprised of from about 0.01 percent to about 95 percent active ingredient; however, powders and tablets of the invention are not limited to these percentages of active ingredient. Suitable solid carriers are known in the art, e.g., magnesium carbonate, magnesium stearate, talc, sugar or lactose. Tablets, powders, cachets and capsules can be used as solid dosage forms suitable for oral administration. Liquid form preparations include solutions, suspensions and emulsions, for example, water or water-propylene glycol solutions for parenteral injection. Solid form preparations may be converted into liquid preparations shortly before use, to liquid form preparations for either oral or parenteral administration. Parenteral forms to be injected intravenously, intramuscularly or subcutaneously are usually in the form of sterile solutions and may contain agents such as salts or glucose, and buffers. Opacifiers may be included in oral solutions, suspensions and emulsions. Liquid form preparations may also include solutions for intranasal administration. Aerosol preparations suitable for inhalation may include solutions and solids in powder form, which may be in combination with a pharmaceutically acceptable carrier, such as an inert compressed gas, for example, nitrogen.

The compositions employed in the present invention may also be deliverable transdermally. The transdermal compositions can take the form of creams, lotions, aerosols and/or emulsions and can be included in a transdermal patch of the matrix or reservoir type as are conventional in the art for this purpose. Examples of pharmaceutically acceptable carriers and methods of manufacture for various compositions are known in the art (see A. Gennaro (ed.), Remington's Pharmaceutical Sciences, 18th Edition, (1990), Mack Publishing Co., Easton, Pa.).

Methods of the present invention are useful for treating cancerous conditions including, but not limited to, those discussed below.

Malignant melanoma is a type of skin cancer and is often characterized by dark and suspicious looking moles. There are definite signs that a mole is suspicious, such as if the mole is getting bigger or is changing shape (e.g., irregular edges) or color (e.g., getting darker, patchy or multi-shaded). Further signs are itching and painful moles, bleeding moles, and inflamed looking moles. The cells that become cancerous in malignant melanoma are called melanocytes. Melanocytes are found between the dermis and epidermis (i.e., the two layers of the skin). Melanocytes produce a pigment or coloring for the skin which helps to protect the body from the ultraviolet radiation of the sun. Melanocytes produce more pigment as the skin is exposed to the sun for longer periods of time and the pigment is transferred to other skin cells to protect them against the ultraviolet radiation.

Some people are more at risk for developing malignant melanoma than others. Risk factors include having lots of moles, being fair skinned, having freckles, being prone to sun burns, on-and-off sun exposure and family history (genetics). In order to test for malignancy, the skin lesions (i.e., the suspicious looking mole(s)) need to be removed for a biopsy. If the mole is found to contain cancerous or pre-cancerous cells then the patient may need a wide local excision of the mole area which includes the removal of some healthy tissue to make sure no malignant cells are left behind. This depends on how much of the mole was left behind and how far the mole has grown into the tissue beneath the skin. Generally, the deeper the cancerous cells have grown into the tissue under the skin, the more likely it is that the melanoma cells have spread to other parts of the body.

If the melanoma is confined to the top layer of the skin (the epidermis) there is usually a low risk of recurrence. If the melanoma has grown into the dermis, there is a medium risk for recurrence. If the melanoma has grown beyond the dermis, into the fat layer under the skin, or even further than that, there is a high risk of recurrence. High risk melanomas often require further tests such as blood tests, chest-x-ray, ultrasound scans, bone scan, and CT scans. Advanced malignant melanomas are characterized by having spread to other parts of the body. Cancer that has spread to another part of the body is generally referred to as secondary cancer or metastases.

Hairy cell leukemia is a disease in which cancer cells are found in the blood and bone marrow. The disease is called hairy cell leukemia because the cancer cells look hairy when examined under a microscope. Hairy cell leukemia affects white blood cells called lymphocytes which are made in the bone marrow and other organs. When hairy cell leukemia develops, the leukemia cells may collect in the spleen causing an enlarged spleen. Furthermore, the blood may contain too few normal white blood cells because the leukemia cells invade the bone marrow and the marrow cannot produce enough normal white blood cells. This can result in a decreased resistance to infections. Most afflicted patients suffer from a gradual onset of fatigue, a spleen that is larger than normal or an infection. The diagnosis usually includes blood tests and a bone marrow biopsy.

Chronic myelogenous leukemia (CML) is a slowly progressive blood and bone marrow disease. CMV is not an inherited disease; however, it is believed to have a genetic component. CML is caused by a change in a chromosome called the Philadelphia chromosome in marrow cells that leads to overproduction of white blood cells. CML usually develops slowly, although it can progress to a fast-growing accelerated phase. CML symptoms, which typically develop gradually, include fatigue, unexplained weight loss, shortness of breath, and a pale complexion due to anemia. A complete diagnosis requires examinations of both blood and marrow cells. The identification of abnormally high numbers of fully matured and maturing white blood cells (i.e., myelocytes and neutrophils) is the first step toward a diagnosis. The diagnosis is confirmed if a marrow sample reveals cells containing the abnormal Philadelphia chromosome. In most CML patients, the chronic phase of the disease transforms into the second phase that becomes more difficult to manage. The second phase is called the accelerated phase. During the accelerated phase, the numbers of white blood cells and immature or blast cells in the bloodstream increase. The third phase is called blast crisis, which is similar to an aggressive acute leukemia. CML is usually diagnosed in the chronic phase.

Kidney cancer often begins with relatively few symptoms. The cancer can be picked up on an ultrasound scan at an early stage of the disease. As the cancer progresses, however, the symptoms become more visible such as blood in the urine, a lump or mass in the area of the kidney, elevated temperature, heavy sweating, loss of appetite, weight loss, a pain in the side that won't subside, tiredness and a general feeling of poor health. High blood pressure and anemia can also be symptoms of kidney cancer. Renal cell cancer is the most common type of kidney cancer in adults. Renal cell cancer is also called renal adenocarcinoma or hypernephroma. In renal cell cancer the cancerous cells are found in the lining of the tubules (the smallest tubes inside the nephrons that help filter the blood and make urine). Other types of kidney cancer are transitional cell cancer and Wilm's tumor. Diagnosis of kidney cancer usually begins with a urine test. Small amounts of blood in the urine (haematuria) can be as sign of kidney cancer. Another test is ultrasound which shows any growth inside the kidney. Intravenous pyelogram (IVP) or urogram (IVU) employs a dye injected into the blood stream to show any growth in the tubes inside or leading from the kidneys. The grade of the cancer depends on the appearance of the cancer cells under the microscope. The more abnormal the cancer cells appear (compared to normal kidney cells) the higher the grade of the cancer. Low grade cancers grow slower and are less likely to spread to other parts of the body than high grade cancers. If the cancer has spread (metastases) to other parts of the body than the patient is usually diagnosed with advanced kidney cancer.

Liver cancer is somewhat rare. Most often liver cancer is the result of other cancers that have spread to the liver from other organs such as breast or bowl, thus, it is referred to as secondary liver cancer. There are certain risk factors associated with primary liver cancer, such as cirrhosis (scarring of the liver due to previous damage), viral hepatitis (chronic infection with hepatitis B or C), aflaxotin (a substance found in moldy peanuts, wheat, soy beans, ground nuts, corn and rice), exposure to chemicals (e.g., vinyl chloride and thorium dioxide), oral contraceptives, anabolic steroids, smoking, chronic inflammatory conditions (e.g., ulcerative colitis) and liver fluke infection (liver fluke is a parasite that can cause a chronic infection). Diagnosis of primary liver cancer usually includes specific blood tests such as liver function tests (LFT); including an alpha-fetoprotein (AFP) test. In patients with hepatoma, AFP is higher than normal. Further tests include an ultrasound scan, CT scan, MRI scan, liver biopsy, laparoscopy (a laparoscope is inserted into the abdomen through a small cut to scan for signs of cancer), hepatic angiography (too visualize the arteries that supply blood to the liver to see how close any tumors are to major blood vessels), x-rays and bone scan (to scan for hepatoma which can spread to the bones).

Bladder cancer can be caused by a number of different chemicals and/or agents but is often caused by cigarette smoking and occupational exposure to a certain class of organic chemicals called aromatic amines (e.g., beta-naphthylamines, xenylamine, 4-nirtobiphenyl, and benzidine). It can take from five to 50 years from the first exposure to a carcinogenic agent until bladder cancer is diagnosed in a subject. Generally, the more cigarette smoking the subject engages in the greater is the risk for developing bladder cancer. The most common clinical sign is blood in the urine, called hematuria. This is often painless and the blood may be visible to the naked eye (gross hematuria) or can be seen only under the microscope (microscopic hematuria). Frequently the diagnosis of bladder cancer is delayed because bleeding is intermittent or attributed to other causes such as urinary tract infection or blood thinners.

The vast majority of bladder cancers are transitional cell cancers. If a urinary cytology is positive, then the patient is very likely afflicted with transitional cell cancer of the urothelium. However, cytologic examinations may be negative in up to half of patients with bladder cancer, thus, a negative finding does not rule out bladder cancer. In addition, one can test for bladder cancer with dip-stick tests of the urine and DNA ploidy analysis (i.e., a method of measuring the amount of DNA in the cancer cells). Since transitional cells line the urinary tract starting at the kidney, down the ureter, into the bladder and includes most of the urethra, the entire urinary tract needs to be evaluated for transitional cell cancer. The lining of the kidneys (renal pelvis) and ureters can be evaluated by intravenous pyelogram (IVP) or retrograde pyelogram.

Non-Hodgkin's lymphoma occurs more frequently than Hodgkin's lymphoma. Non-Hodgkin's lymphomas can be slow-growing (i.e., low-grade) or rapidly growing (i.e., high-grade) cancers. The symptoms are mainly enlarged lymph nodes (such as an armpit lump), fever, excessive sweating, and weight loss. For most patients, the cause is unknown, but non-Hodgkin's lymphomas can develop in people with a suppressed immune system, such as after organ transplantation. Thus, high-risk groups include organ transplant recipients and immunosuppressed people. Most often non-Hodgkin's lymphomas affect people older than 50 years. There are a number of tests that can indicate non-Hodgkin's lymphoma which include a peripheral blood smear showing abnormal white blood cells, a lymph node biopsy and a bone marrow biopsy. In order to tell the extent of the tumor (i.e., the stage of the tumor) certain physical evaluations may be conducted such as CT scans of the chest, abdomen and pelvis; a lymphangiogram; an exploratory laparotomy or liver biopsy; blood chemistry tests; MRI or other X-ray studies and a PET (positron emission test) scan.

Hodgkin's lymphoma is a malignancy (cancer) of lymphoid tissue found in the lymph nodes, spleen, liver, and bone marrow. The major symptoms of this disease are painless swelling of the lymph nodes in the neck, armpits, or groin (swollen glands), fatigue, fever, chills, night sweats, weight loss, loss of appetite and generalized itching. The disease may be diagnosed via a lymph node biopsy, a bone marrow biopsy, a biopsy of suspected tissue and detection of Reed-Sternberg (Hodgkin's lymphoma) cells by biopsy. A staging evaluation (tumor staging) to determine extent of disease includes, but is not limited to, CT scans of the chest, abdomen and pelvis; a bone marrow biopsy; blood chemistry tests; a PET scan and an abdominal surgery to biopsy the liver and spleen. A staging evaluation is also necessary to determine the treatment plan. For example, stage I indicates one lymph node region is involved (e.g., the right neck). Stage II usually indicates involvement of two lymph nodes on the same side of the diaphragm (e.g., both sides of the neck). Stage III indicates lymph node involvement on both sides of the diaphragm (e.g., groin and armpit). Stage IV involves a tumor that has spread outside the lymph nodes (e.g., to bone marrow, lungs, and/or liver).

Kaposi's sarcoma is a malignant tumor of the connective tissue, often associated with AIDS. Associated symptoms are bluish-red nodules (macule and/or papule) with an irregular shape, bleeding with gastrointestinal lesions, shortness of breath with pulmonary (lung) lesions, and bloody sputum with pulmonary lesions. Early lesions (i.e., nodules) may start on the feet or ankles and spread to the arms and hands. Kaposi's sarcoma can be seen in the elderly where it tends to develop slowly. In patients with AIDS, the disease tends to develop aggressively and involves the skin, lungs, gastrointestinal tract, and other organs. In people with AIDS, Kaposi's sarcoma is caused by an interaction between HIV, immune system suppression, and human herpesvirus-8 (HHV-8). The diagnosis for this disease includes various tests such as a skin lesion biopsy that shows Kaposi's sarcoma and an endoscopy that shows Kaposi's lesions. Sometimes this disease alters the results of an esophagogastroduodenoscopy (EGD) which is a test that involves visually examining the lining of the esophagus, stomach, and upper duodenum with a small camera (flexible fiber optic endoscope).

The present invention provides treatment for various cancerous conditions including, but not limited to, skin cancer (e.g., melanoma), leukemia (for example, and without limitation, hairy cell leukemia and chronic myeloid leukemia), kidney cancer, liver cancer, bladder cancer, lymphoma and Kaposi's sarcoma. In one embodiment, the pharmaceutical compositions are administered systemically to the subject. In another embodiment, the pharmaceutical compositions are administered orally, nasally or through injection (e.g., subcutaneous, intramuscular, intravenous, supra). In another embodiment, the therapeutically effective amount of the pharmaceutical compositions can be administered in a dose ranging from about 0.1 to about 100 million IU. In still another embodiment, the therapeutically effective amount of the pharmaceutical compositions can be administered in a dose ranging from about 1 to about 50 million IU. The administration of the therapeutically effective amount of the pharmaceutical compositions can occur about one time per week or more often or less often. Alternatively, the administration of the therapeutically effective amount of the pharmaceutical compositions can occur less often than one time per week.

Immunotherapy used in malignant melanoma employs interferon. Malignant melanoma can be treated with glycosylated interferon alpha, particularly when the disease has spread to the lymph nodes. As such, melanoma can be treated with a pharmaceutical composition of glycosylated interferon alpha, for example, at a dose from about 0.1 to about 100 million IU once per week or more often or less often depending on the severity of the melanoma. The treatment is often given as an injection under the skin (subcutaneous injection). The injections can be given, for example, three times a week. Some treatment plans include interferon given daily for the first few weeks. The daily injections can be given into a vein (intravenous injection) rather than under the skin. Interferon treatment may continue for several years depending on the progress of the disease. Glycosylated interferon alpha, as disclosed herein, can also be administered in combination with chemotherapy and/or radiation therapy. Particularly, high risk melanomas and advanced malignant melanomas can be treated with a combination of chemotherapy, radiotherapy and/or immunotherapy (i.e., interferons).

Hairy cell leukemia can be treated with glycosylated interferon alpha, for example, at a dose from about 0.1 to about 100 million IU once per week or more often or less often. Hairy cell leukemia can also be treated with glycosylated interferon alpha. As disclosed herein, for example, at a dose from about 0.1 to about 100 million IU once per week or more often or less often in combination with cladribine (2-chlorodeoxyadenosine; CdA) at a dose of, for example, and without limitation, about 0.14 mg per kg daily for 7 days.

Treatment of CML with chemotherapy can induce long periods of remission (i.e., periods when white blood cell counts and symptoms are reduced), however, it does not cure the disease. Treating CML with stem cell transplantation holds the potential for a cure but it is usually accompanied with a high dose of chemotherapy or radiation. A better form of treatment employs glycosylated interferon alpha, as disclosed herein. As such, CML can be treated with glycosylated interferon alpha, for example, at a dose from about 0.1 to about 100 million IU once per week or more often or less often. Treatment with glycosylated interferon alpha provides a low risk treatment that is often the best for patients with poor physical health who would be unlikely to tolerate the high-dose chemotherapy and/or radiation therapy that accompanies stem cell transplantation. CML may also be treated with glycosylated interferon alpha, for example, at a dose from about 0.1 to about 100 million IU once per week or more often or less often in combination with chemotherapy/radiation and/or stem cell transplantation.

The treatment of kidney cancer depends on how far advanced the cancer is. Early stage kidney cancer can be treated with surgery (removal of a kidney or parts of a kidney) as long as the cancer has not spread locally (i.e., to tissues next to the affected kidney) or to other parts of the body. However, if the cancer has spread locally or to other parts of the patient's body or the patient cannot tolerate the invasive surgery, treatment with glycosylated interferon alpha, as disclosed herein, is preferred.

Early stage kidney cancer can be treated with glycosylated interferon alpha, for example, at a dose from about 0.1 to about 100 million IU once or more times per week. Treatment with glycosylated interferon alpha can be combined with surgery (interferon is administered after surgery to prevent the tumor from coming back), radiation therapy (using high energy rays that destroy the cancer cells), arterial embolisation (cuts off the tumor's main blood supply), cryotherapy (freezes the tumor), radio-frequency-ablation (killing the tumor with heat), and high-intensity ultrasound (HIFU) (also killing the tumor with heat).

Advanced kidney cancer (i.e., cancer that has spread locally or to other parts of the body) is preferably treated with a dose of glycosylated interferon alpha, for example, from about 0.1 to about 100 million IU once or more times per week. Interferon may also be administered three times per week as a small injection under the skin. Paracetamol may be taken about a half hour before the treatment with interferon and every six hours after the treatment to prevent or reduce any possible side effects such as fever, chills, headache, backache and joint pain. Once the side effects subside paracetamol is no longer administered. Injections can be given by a physician, nurse, patient aid, or the patient him or herself. Treatment with glycosylated interferon alpha is particularly effective against advanced kidney cancer that has spread to the lungs and other organs. Advanced kidney cancer can also be treated with glycosylated interferon alpha in combination with surgery, arterial embolisation, radiotherapy, chemotherapy, and biological therapy (e.g., with other biological agents such as interleukin 2).

Primary liver cancer can be treated with glycosylated interferon alpha or glycosylated interferon alpha in combination with surgery, chemotherapy, and/or sometimes radiation therapy. Many hepatomas (i.e., most common type of primary liver cancer) are not removable by surgery because they are not contained in one area of the liver. Thus, administering glycosylated interferon alpha to the patient provides an alternative treatment that is promising and much less invasive. When the tumor cannot be removed it can also be treated with a combination of chemotherapy and/or radiation therapy and/or glycosylated interferon alpha. Liver cancer can be treated with a dose of glycosylated interferon alpha from about 0.1 to about 100 million IU once or more times per week. Furthermore, glycosylated interferon alpha can be used to treat hepatoblastoma (liver cancer that affects young children) alone or in combination with chemotherapy and/or radiation therapy.

Bladder cancer can be of a superficial or invasive type. Patients with small, single low-grade tumor with a normal amount of DNA (diploid) in the cancer cells that are limited to the urothelium are generally at low risk for recurrence. If the tumor recurs, the patient can be treated with glycosylated interferon alpha at a dose from about 0.1 to about 100 million IU, or at a dose ranging from about 0.1 to about 50 million IU once or more times per week.

Patients with multiple tumors, high-grade, abnormal amounts of DNA ploidy (aneuploid), with carcinoma in situ or tumor penetration into the lamina propria are at high risk for tumor recurrence and progression. Usually, random bladder biopsy specimens and cytologic examinations reveal abnormalities. Glycosylated interferon alpha can be administered at a dose from about 0.1 to about 100 million IU once time per week or more often or less often for high risk patients. Occasionally, long-term maintenance glycosylated alpha interferon treatment regimens can be employed. For invasive bladder cancer, additional treatment may be employed such as irradiation, systemic chemotherapy, surgery, treatment with glycosylated interferon alpha or any combination thereof. Although radiation therapy alone allows the bladder to be preserved, the five-year survival for patients with tumors into the innermost part of the muscle layer of the bladder is about 40 percent, into the deep muscle layer or just beyond the muscle layer is about 20 percent, and into adjacent organs (prostate or vagina) is about 10 percent (see Wesson M. F. (1992) Urologic Clinics of North America, 19(4):725-734). Thus, treatment with glycosylated interferon alpha provides new hope for this patient population. Glycosylated interferon alpha may also be used in conjunction or in combination with radiation therapy and intravenous chemotherapy.

Although, for invasive cancer that appear to be within the bladder, complete surgical removal of the bladder provides the best chance of a cure, a partial removal of the bladder may be tried in some patients. This has the advantage of preserving the bladder and sexual function as well as quality of life. Patients with only one tumor located near the dome of the bladder and without carcinoma in situ in other areas of the bladder, are the best candidates for partial bladder removal. Treatment with glycosylated interferon alpha can be used to treat these patients at a dosage from about 0.1 to about 100 million IU once or more times per week at the time of partial bladder removal and/or after partial bladder removal.

The treatment of non-Hodgkin's lymphoma depends upon the stage of the disease. A low-grade lymphoma may need to be observed until it causes problems. When treatment becomes necessary, the patient can be treated with glycosylated interferon alpha, chemotherapy or radiation therapy or combinations thereof. Treatment with glycosylated interferon alpha can be used to treat patients at a dosage from about 0.1 to about 100 million IU once or more times per week. Patients with more aggressive or resistant disease may require more intensive treatment and higher doses of interferon. High-dose chemotherapy in combination with interferon and bone marrow transplantation can be a treatment option in selected cases where the tumor is particularly aggressive.

The treatment of Hodgkin's lymphoma varies with the stage of the disease. Stages I and II (limited disease) can be treated with glycosylated interferon alpha, localized radiation therapy, chemotherapy or with combinations thereof. Treatment with glycosylated interferon alpha can be used to treat these patients at a dosage from about 0.1 to about 100 million IU once or more times per week. Stages III and IV (extensive disease) can be treated with glycosylated interferon alpha (higher doses if necessary) and/or a combination of radiation therapy and chemotherapy. Chemotherapy can cause low blood cell counts, which can lead to an increased risk of bleeding, infection, and anemia. Advantageously, treatment with glycosylated interferon alpha can eliminate or reduce the amount of chemotherapy and/or radiation therapy necessary.

The treatment of Kaposi's sarcoma depends on the extent and location of the lesions, as well as the subject's symptoms and degree of immunosuppression. Treatment with glycosylated interferon alpha can be used to treat patients at a dosage from about 0.1 to about 100 million IU once or more times per week depending on the extent of the tumor. Radiation therapy and/or cryotherapy with or without interferon can be used for lesions in certain areas. Combinations of chemotherapy and interferon may also be used. Since lesions often recur after treatment with radiation and cryotherapy, the treatment with glycosylated interferon alpha alone or in combination with other therapies holds great promise. Antiretroviral therapy in combination with glycosylated interferon alpha can further shrink the lesions in AIDS patients.

Methods of the present invention are useful for treating viral conditions including, but not limited to, those discussed below.

The term hepatitis refers to syndromes or diseases that cause liver inflammation, including inflammation due to viruses and chronic alcohol abuse. Viruses causing hepatitis include Hepatitis A, B, C, E and the delta factor. The present invention also contemplates the treatment of each of the genotypes of hepatitis, including, but not limited to, hepatitis C genotype 1, hepatitis C genotype 2 and hepatitis C genotype 4. Each virus causes a distinct syndrome, although the viruses share some symptoms and consequences. A short infection of the hepatitis B (HBV) or hepatitis C virus (HCV) is known as an acute case of hepatitis B or hepatitis C, respectively. People infected with the hepatitis virus may develop a chronic, life-long infection. Such individuals may show symptoms; however, many of these patients never develop symptoms and are known as carriers, wherein they can spread the disease to others. Chronic hepatitis increases a patient's chance of developing permanent liver damage, including cirrhosis (scarring of the liver) and liver cancer. Hepatitis can be transmitted via blood and other bodily fluids. Infection can occur through contact with blood in healthcare settings; unsafe sex with an infected person; blood transfusions; the sharing of needles during drug use; tattoo or acupuncture with contaminated instruments; and birth (i.e., an infected mother can transmit the virus to the baby during delivery or shortly thereafter). Symptoms of hepatitis include fatigue, malaise, joint aches (arthralgias), low grade fever, nausea, vomiting, loss of appetite, abdominal pain, and jaundice and dark urine due to increased bilirubin.

There are a number of tests available that can help to diagnose this disease. For example, in case of hepatitis B, certain specific tests are available such as testing for hepatitis B surface antigen (HBsAg) which represents the first viral marker present in blood tests after the patient has been infected. HBsAg usually disappears from the blood in one to two months. Diagnosis further includes testing for hepatitis B core antibody (Anti-HBc) which is usually detected within 1-2 weeks of the appearance of hepatitis B surface antigen. Diagnosis still further includes testing for hepatitis B surface antibody (Anti-HBs) which is found both in those who have been immunized and those who have recovered from hepatitis infection; and testing for both hepatitis B surface antibody and core antibody which persist indefinitely in the blood of patients who have recovered from hepatitis B. Another test includes examining liver enzyme (transaminase) blood levels which may be elevated due to liver damage. Also, albumin levels may also be tested. Albumin may be low and prothrombin time may be prolonged due to severe liver failure. Tests for hepatitis C include hepatitis virus serology with negative antibody to hepatitis A and hepatitis B; an ELISA assay to detect hepatitis C antibody; a hepatitis C PCR test; testing for elevated liver enzymes; a liver biopsy which shows acute or resolving hepatitis; and a hepatitis C genotype. Six genotypes are present around the world and most Americans are afflicted with a genotype I infection, which has lower response rates to treatment.

Venereal or genital warts are soft wart-like growths on the genitals caused by a viral skin disease. Genital warts are transmitted via sexual intercourse (known as a sexually transmitted disease or STD) and are caused by the Human papilloma virus (HPV). Symptoms are not always present in infected individuals. However, some associated symptoms include, but are not limited to raised, flesh-colored lesions on the genitals, anus, or surrounding skin; cauliflower-like appearing growths around the anus or genitals; increased dampness or moisture in the area of the growths; and itching of the genital areas. Diagnosis includes a physical examination that reveals flesh-colored to white, flat or raised, single or clustered lesions anywhere on the genitalia; a pelvic examination in women may reveal growths on the vaginal walls or the cervix; and a pap smear in women may note changes associated with HPV.

Measles or rubeola is a highly contagious viral disease characterized by fever, cough, conjunctivitis (redness and irritation in membranes of the eyes), and a rash that spreads across the body. Measles is caused by a virus and the infection is spread by contact with droplets from the nose, mouth, or throat of an infected person. The incubation period is usually 8 to 12 days before symptoms first appear. Immunity to the disease occurs after vaccination or active infection. Most children are vaccinated against the disease via the MMR vaccine, which protects against measles, mumps, and rubella. Although, some parents refuse the vaccination due to fear that it may can cause autism in some children. Thus, lower vaccination rates can cause outbreaks of measles which can be quite serious especially if older people are infected with the virus. The diagnosis of measles includes a viral culture which is rarely done and a measles serology (i.e., a blood test to detect the presence of antibodies against a microorganism).

The present invention contemplates treatment for various viral conditions including, but not limited to, hepatitis B, hepatitis C, venereal warts and measles via pharmaceutical compositions of glycosylated interferon alpha. The viral condition may be a chronic condition. The methods of the present invention can be employed to treat a subject that is a patient infected with hepatitis C virus (HCV) or a patient infected with HCV and further infected with human immunodeficiency virus (HIV). In order to treat a subject for a viral condition, different routes of administration may be selected. In one embodiment, the pharmaceutical compositions are administered systemically (e.g., sustained release, implants, injections) to the subject. In another embodiment, the pharmaceutical compositions are administered locally such as orally (e.g., capsules, tablets), nasally (e.g., nasal sprays) or through injection (e.g., subcutaneous or intramuscular or intravenously). In another embodiment, the therapeutically effective amount of interferon alpha can be administered in a dose ranging from about 0.1 to about 100 million IU. In still another embodiment, the therapeutically effective amount of the interferon alpha can be administered in a dose ranging from about 1 to about 50 million IU. The administration of the therapeutically effective amount of the interferon alpha can occur about one time per week. Alternatively, the administration of the therapeutically effective amount of the interferon alpha can occur more or less often than one time per week. For treatment of a viral condition, the interferon alpha may be administered in combination with at least one additional agent. Such agents include, but are not limited to, viramidine and ribavirin. The agents may be administered simultaneously with the glycosylated interferon, for example, in the same pharmaceutical composition, or sequentially.

The treatment of hepatitis B depends on the stage of the disease. Acute hepatitis B needs careful monitoring of the liver function, by measuring serum transaminases and prothrombin time. In cases of liver failure, the patient should be monitored in an intensive care unit and treated with glycosylated interferon alpha. The patient can be administered a dose ranging from about 0.1 to about 100 million IU of the interferon one time per week or more often or less often than one time per week depending on the disease progression. The glycosylated interferon alpha can be administered, for example, by subcutaneous or intramuscular injection. Because damage to the liver decreases its ability to degrade proteins, protein intake should be restricted and oral lactulose or neomycin can be administered (to limit protein production by bacteria in the gut). Patients should be monitored until they recover. Treatment of chronic hepatitis B is geared towards reducing inflammation, symptoms, and infectivity. Treatment with glycosylated interferon alpha may convert about 30-40 percent of patients from the replicative phase to non-replicative phase. Similarly, the patient can be administered a dose ranging from about 0.1 to about 100 million IU of a pharmaceutical composition of glycosylated interferon alpha one time per week or more or less often than one time per week. The pharmaceutical composition can be administered, for example, by subcutaneous or intramuscular injection. Sometimes the drug may cause some adverse side effects which include a flu-like syndrome, fever and chills. However, these side effects are fewer and less severe than with standard recombinant interferon alpha (e.g., Intron®-A interferon by Schering-Plough). End-stage chronic hepatitis B liver disease can be treated with a combination of liver transplantation and glycosylated interferon alpha.

Another aspect of the present invention provides methods for treating a viral condition in a subject including administering to the subject a therapeutically effective amount of a pharmaceutical composition comprising glycosylated interferon alpha in combination with ribavirin or viramidine. For example, and without limitation, glycosylated interferon alpha 2b in combination with ribavirin or viramidine is contemplated by the present invention. Other agents, pharmaceutical carriers and/or excipients may further be included in the pharmaceutical compositions.

The treatment of hepatitis C includes administering to the patient a pharmaceutical composition of glycosylated interferon alpha with a dose ranging from about 0.1 to about 100 million IU one time per week or more or less often than one time per week. Patients with hepatitis C also benefit from treatment with a combination of glycosylated interferon alpha and ribavirin. For example, subjects can be administered an amount of ribavirin in accordance with the present invention in an amount in a range of between about 10 milligrams and about 10 grams per administration. The administrations may be one or more times per day or fewer that one time per day. In one embodiment, patients can be administered 1200 mg per day of ribavirin plus 3 million IU of glycosylated interferon alpha three times per week. Glycosylated interferon alpha can be given, for example, by injection just under the skin. Advantageously, the injections can be administered less often relative to standard recombinant interferon alpha, when administered with ribavirin.

Ribavirin (e.g., Rebetol® medication by Schering-Plough) is a capsule or tablet that can be taken twice daily; it is often administered at 800-1200 mg per day (see PWA Health Group, Ribavirin Info Sheet). Ribavirin is also available in aerosol form. Glycosylated interferon alpha and ribavirin can lead to a sustained response in the majority of patients (more than about 50 percent). This means that the patient remains free of hepatitis C virus for at least six months after stopping therapy.

Similarly, such patients may also benefit from treatment with a combination of glycosylated interferon alpha and viramidine. Viramidine is a prodrug of ribavirin which is converted by adenosine deaminase to ribavirin in liver cells. Compared with ribavirin there is less uptake of viramidine into red blood cells, which may be associated with a reduction in haematological toxicity, due to the positive charge on the viramidine molecule. Viramidine has been shown to be safe and well tolerated with single doses of up to 1200 mg per day (see Hep. Dart. (2003), Frontiers in Drug Development for Viral Hepatitis, Internet Conference Report, Dec. 14-18, 2003, Hawaii) and is available in oral form (e.g., as Viramidine™ medication from Valeant Pharmaceuticals International). Besides treatment with glycosylated interferon, people with hepatitis C should avoid any substances toxic to the liver (hepatotoxic) including alcohol and some vitamins.

The treatment of venereal or genital warts may include a local application of a medication to the skin. Such a medication can include, for example, administering to the patient's skin (e.g., by injection) a pharmaceutical composition of glycosylated interferon alpha with a dose ranging from about 0.1 to about 100 million IU one time per week or more or less often than one time per week. The pharmaceutical composition of interferon may also be injected alone or in combination with other agents. Surgical treatments include cryosurgery, electro-cauterization, laser therapy, or surgical excision alone or in combination with glycosylated interferon alpha. Patients have to be monitored and should schedule follow-up visits with their physician to detect any recurrence of the disease. If the disease recurs, larger doses of glycosylated interferon alpha can be administered to prevent further recurrence.

Measles can be treated by administering to the patient a pharmaceutical composition of glycosylated interferon alpha with a dose ranging from about 0.1 to about 100 million IU one time per week or more or less often than one time per week. The pharmaceutical composition of interferon may be administered alone or in combination with other agents. Besides interferon, there is no other specific treatment of measles, though some children may require supplementation with Vitamin A. Since faster symptomatic relief may be achieved with glycosylated interferon than with bed rest, glycosylated interferon holds great promise to treat infected populations as the spread of measles can be reduced or completely diminished.

The amount and frequency of administration of the pharmaceutical compositions of the invention and/or the accompanying agents will be regulated according to the judgment of the attending clinician considering such factors as age, condition and size of the patient as well as severity of the symptoms being treated.

The following specific examples are intended to illustrate the invention and should not be construed as limiting the scope of the claims. The glycosylated alpha interferons employed in the examples are human alpha interferons which are produced in transgenic animals (i.e., transgenic avians, e.g., transgenic chickens). Pharmaceutical compositions employed in administering the interferon in the examples can typically include 7.5 mg/ml NaCl, 1.8 mg/ml sodium phosphate dibasic, 1.3 mg/ml sodium phosphate monobasic, 0.1 mg/ml edetate disodium, 0.1 mg/ml polysorbate 80, and 1.5 mg/ml m-cresol.

### EXAMPLE 1

### Treatment of Melanoma with Glycosylated Interferon Alpha 2b

A patient presents with a suspicious looking mole that appears to be getting bigger on a monthly basis. The tending physician closely inspects the mole and notices irregular edges and a color that appears patchy and multi-shaded. The mole appears inflamed and slightly raised. The patient further complains of pain and itching around the mole area. The patient is a 34 year old caucasian female with fair skin and multiple freckles in the areas of face and neck. The mole is removed under local anesthetic treatment and sent to the laboratory for a biopsy. The biopsy reveals cancerous cells and the patient is further subjected to a local excision of the mole area under complete anesthesia. The surgery reveals that the mole has grown into the dermis and beyond the dermis into the fat layer under the skin. Since the patient is at medium to high risk for metastases, the physician further subjects the patient to additional blood tests and a CT scan. The CT scan reveals that the cancer has spread to the lymph nodes but not to the liver or other organs.

The patient is subjected to a treatment of a pharmaceutical composition of glycosylated interferon alpha 2b of 27 million IU three times per week to stop the spread of the cancer. The pharmaceutical composition is administered as a subcutaneous injection under the skin by the physician. The patient is closely monitored and a second CT scan is performed after 2 weeks of continuous treatment with IFN alpha 2b. The second CT scan reveals that the tumor has not spread beyond the lymph nodes and appears to be contained in specific areas within the lymph nodes. A bone scan confirms that the tumor has not spread to the bones. The treatment regimen continues for an additional two weeks and is then lowered to 20 million IU of IFN alpha 2b two times per week via subcutaneous injection for a period of two months. After the two month treatment period, the patient is subjected to further blood tests, a CT scan, a bone scan, a chest-x-ray, and an ultrasound scan of the lymph nodes and liver. The blood, lymph nodes and liver appear clear of metastases. No further spread of the cancer is detected via CT scan and the patient is administered a lower treatment of IFN alpha 2b, of 10 million IU of IFN alpha 2b one time per week via subcutaneous injection for a period of one month. A follow-up visit reveals that the patient is stabilized and the treatment is lowered to maintain the current status. The patient is administered a dosage of 2 million IU of IFN alpha 2b one time per week via subcutaneous injection for a period of one month. A follow-up visit reveals that the patient remains stable and a maintenance dosage of 0.3 million IU of IFN alpha 2b one time every four weeks via subcutaneous injection for a period of five month is prescribed. The patient will be continuously monitored and remain on this dosage for a period of one year. If there is no recurrence of the cancer after one year, the treatment of IFN alpha 2b can be discontinued. If the cancer recurs, the original treatment can be reinitiated. The patient has the option to remain on a maintenance dose of IFN alpha 2b to prevent a further recurrence. No adverse side effects are experienced by the patient during the treatment period.

### EXAMPLE 2

### Treatment of Hepatitis C with a Combination of Glycosylated Interferon Alpha 2b and Ribavirin

A patient presents with characteristic symptoms of hepatitis C including fatigue, joint aches, low grade fever and abdominal pain. The patient is a 42 year old caucasian male. A urine test reveals increased bilirubin levels. Tests for hepatitis B surface antigen (HBsAg) and hepatitis B core antibody (Anti-HBc) are negative. An ELISA assay for hepatitis C antibody and a hepatitis C PCR test are positive for hepatitis C genotype 1. qRTPCR reveal a viral load in excess of 1,000,000 copies per ml of serum. A blood serology shows elevated liver enzymes. As a result of the initial findings, the physician subjects the patient to a liver biopsy to test for acute hepatitis which is confirmed.

Due to already existing moderate liver damage, the patient is subjected to a low protein diet and is carefully monitored. The patient is then subjected to a treatment of a pharmaceutical composition of glycosylated interferon alpha 2b of 3.4 million IU one time per week by an injection just under the skin. In addition, the patient is co-administered 1100 mg per day of oral ribavirin (Rebetol® medication by Schering-Plough) in soft capsule form. This combination therapy continues for a period of two months while the patient is under close observation by the responsible physician (i.e., continuous visits on an out-patient basis). At the end of the two month treatment period, a blood serology of the patient is reassessed and it reveals normal amounts of liver enzymes. The physician downgrades the patient's condition to resolving hepatitis C and the treatment is continued at a lower dosage of 1.2 million IU of glycosylated IFN alpha 2b one time per week by an injection just under the skin for an additional two month in combination with 800 mg per day of oral ribavirin. After 4 months of treatment, the patient's viral load is determined by qRTPCR to be less than 100 copies per ml of serum.

The patient will be reassessed after one year. Another liver biopsy is recommended within a period of two years if any characteristic symptoms of hepatitis C reappear. No adverse side effects are experienced by the patient during the treatment period.

### EXAMPLE 3

### Treatment of Genital Warts with Glycosylated Interferon Alpha 2b

A woman of 38 years has a history of 6-monthly cervical smears showing dysplasia for 20 years, during which time she has borne 3 children and receives local laser therapy. Following a single epidermal injection of 200,000 IU of glycosylated interferon alpha 2b, her next 2 regular cervical examinations are normal for the first time in over 20 years.

### EXAMPLE 4

### Treatment of Warts with Glycosylated Interferon Alpha 2b

A 12 year old boy has a large, brownish colored plantar wart on the upper inside portion of the heel of his right foot. For more than two years, the wart has caused the boy mild, to extreme discomfort, particularly when walking. On numerous occasions over the course of this period, the wart has been treated with several over the counter, and physician prescribed topical medications that yield no significant improvement in the condition. The boy is accustomed to shaving the wart off at the skin surface at regular intervals to reduce the size. When the upper layers of the wart are removed in this manner, the remainder of the tumor appears as a cluster of milky-white stones buried below the translucent layers of skin on the heel. Regardless of the treatment employed, the wart continues to grow back and causes discomfort. The wart occupies a brownish, rough area of the heal approximately 25 mm. (1″) in diameter at the base. The central portion is raised, forming a nodule approximately 3 mm above the skin surface.

The wart is treated with injection of 100,000 IU of glycosylated interferon alpha 2b into the skin surrounding the wart (within 5 millimeters of the wart).

Eight weeks after this single treatment, the boy's heel is again examined, and the wart is completely disappeared. Close examination of the entire foot shows no evidence of warts. The previously infected area of skin looks normal and healed, with no trace of disease, or abnormality.

### EXAMPLE 5

### Treatment of Multiple Sclerosis with Glycosylated Interferon Alpha

A 26 year old woman has an attack of transverse myelitis at the lower thoracic level, with a paraparesis involving marked weakness and numbness of both legs. This gradually clears over a two month period. Four months after the first attack, she has a second episode of transverse myelitis at the cervical level, with symptoms involving her arms and legs, and the diagnosis of multiple sclerosis is made. This gradually clears. An attack of transverse myelitis at the lower thoracic level occurs four months later, her third attack in eight months. As she is recovering from this attack, she started on glycosylated interferon alpha 2b administration at a dosage of about 3,800,000 IU one time per week for two months. The patient has no attacks in two years since beginning treatment. No adverse side effects are experienced by the patient during the treatment period.

### EXAMPLE 6

### Treatment of Prostate Cancer with Glycosylated Interferon Alpha 2b

A 70-year-old male is diagnosed with localized prostate cancer (i.e., no metastasis can be detected by means of radionuclide scan and CAT scans). The estimated size of the tumor in the prostate is 7 grams. The location of the tumor is in the right lateral lobe as seen in the sonogram. The PSA level in the blood is 40 ng/ml.

The patient is prepared for a cystoscopy procedure using local anesthesia. A cystoscope is placed into the urethra and the injection needle is inserted into the lesion (the tumor) and is monitored by the ultrasound imaging. Once the needle is secured, 15,000,000 IU of glycosylated interferon alpha 2b is injected slowly over a period of about 4 to 5 minutes. During the injection, vital signs are monitored for symptoms of toxic, allergic, or other adverse reactions.

The patient's recovery is normal, and no signs or symptoms of any adverse reactions to the injected composition are observed. The clinical progress is uneventful. The blood PSA level gradually declines over a period of three months to normal levels (4 ng/ml or less) and remains normal. After 5 years, the patient is diagnosed as free of prostate cancer.

### EXAMPLE 7

### Treatment of Prostate Cancer with Glycosylated Interferon Alpha 2a

A 65-year-old male is diagnosed with localized prostate cancer (i.e., no metastasis can be detected by means of radionuclide scan and CAT scans). The estimated size of the tumor is 15 grams and the total prostate weight is 90 grams. The PSA level in the blood is 200 ng/ml. The tumor is located in the right lobe and is one solid nodule.

The patient is prepared for a cystoscopy procedure using local anesthesia. A cystoscope is placed into the urethra and the injection needle is inserted into the lesion (the tumor) and is monitored by the ultrasound imaging. Once the needle is secured approximately 22,000,000 IU of glycosylated interferon alpha 2a is injected slowly over a period of about 20 minutes. Upon completion of injection, vital signs are monitored for symptoms of toxic, allergic, or other adverse reactions.

The patient's recovery and the post-injection progress is uneventful. The blood PSA level gradually declines to normal levels (4 ng/ml or less) over a period of three months and remains normal. After 5 years, the patient is diagnosed as free of prostate cancer.

### EXAMPLE 8

### Treatment of Liver Cancer with Glycosylated Interferon Alpha

A 59 year old male with a laryngeal epidermoid (squamous type tumor), seven years following treatment in its primary state with radiation therapy, develops metastatic disease in the liver. Two major tumors are noted at a size of 12 cm and 6 cm diameter. The tumors are treated with administration of 32,000,000 IU of glycosylated interferon alpha 2b one time per week for five weeks. This is achieved without any adverse effects and the patient is reassessed four weeks later. At that point in time the smaller tumor has disappeared entirely. The larger tumor is apparent only as a necrotic focus measuring now 5 cm in diameter but no apparent surviving tumor could be detected by examination and needle biopsy.

### EXAMPLE 9

A 53 year old man with transitional cell cancer of the bladder in a very advanced state of his disease with metastases involving the entire left pelvis, extending to the periaortic and parapancreatic and supraclavicular nodes having recurred after previous surgical excision, radiation and having not responded by major regression to standard chemotherapy is treated with a direct injection of 44,000,000 IU of glycosylated interferon alpha 2b into the tumor tissue. The patient develops a febrile reaction due to tumor break down and release of bacteria. This reaction is controlled by antibiotics and appropriate hydration and as the patient is observed through this phase a decrease in the size of the tumor is seen with an over 50% reduction in tumor size occurring by 7 days of therapy.

All references cited herein are incorporated by reference herein in their entirety and for all purposes to the same extent as if each individual publication, patent or patent application is specifically and individually indicated to be incorporated by reference in its entirety for all purposes.

The citation of any publication is for its disclosure prior to the filing date and should not be construed as an admission that the present invention is not entitled to antedate such publication by virtue of prior invention.

Various modifications and variations of the present invention will be apparent to those skilled in the art without departing from the scope and spirit of the invention. Although the invention has been described in connection with specific embodiments, it should be understood that the invention as claimed should not be unduly limited to to such specific embodiments. Indeed, various modifications of the described modes for carrying out the invention which are obvious to those skilled in the art are intended to be within the scope of the claims.

What is claimed is:

1. A method for treating a condition in a subject comprising administering to the subject a therapeutically effective amount of glycosylated interferon alpha.

2. The method of claim 1 comprising monitoring the subject to detect any amelioration of the condition.

3. The method of claim 1 wherein the interferon alpha is interferon alpha 2.

4. The method of claim 3 wherein the interferon alpha 2 is interferon alpha 2b.

5. The method of claim 1 wherein the interferon is present in a pharmaceutical composition.

6. The method of claim 5 wherein the pharmaceutical composition excludes interferon other than glycosylated interferon alpha 2b.

7. The method of claim 1 wherein the interferon is administered systemically to the subject.

8. The method of claim 1 wherein the interferon is administered orally, nasally or through injection.

9. The method of claim 8 wherein the injection is subcutaneous or intramuscular.

10. The method of claim 1 wherein the therapeutically effective amount is a dose ranging from about 0.1 to about 100 million IU.

11. The method of claim 1 wherein the therapeutically effective amount is a dose ranging from about 1 to about 50 million IU.

12. The method of claim 1 wherein the administration of the therapeutically effective amount of the interferon occurs less than one time per week.

13. The method of claim 1 wherein the condition is not substantially ameliorated by administering an interferon alpha that is non-glycosylated.

14. The method of claim 1 wherein the interferon is present in a pharmaceutical composition.

15. The method of claim 14 wherein the pharmaceutical composition comprises a pharmaceutical carrier.

16. The method of claim 14 wherein the pharmaceutical composition excludes interferon other than glycosylated interferon alpha.

17. The method of claim 1 wherein the glycosylated interferon alpha is poultry derived.

18. A method for treating a cancerous condition in a subject comprising administering to the subject a therapeutically effective amount of glycosylated interferon alpha.

19. The method of claim 18 comprising monitoring the subject to detect any amelioration of the cancerous condition.

20. The method of claim 18 wherein the interferon alpha 2 is interferon alpha 2b.

21. The method of claim 18 wherein the interferon is present in a pharmaceutical composition.

22. The method of claim 21 wherein the pharmaceutical composition excludes interferon other than glycosylated interferon alpha.

23. The method of claim 18 wherein the cancerous conditions is selected from the group consisting of skin cancer, leukemia, kidney cancer, liver cancer, bladder cancer, lymphoma and Kaposi's sarcoma.

24. The method of claim 18 wherein the cancerous conditions is melanoma.

25. The method of claim 18 wherein the cancerous conditions is selected from the group consisting of hairy cell leukemia and chronic myeloid leukemia.

26. The method of claim 18 wherein the cancerous condition is not substantially ameliorated by administering an interferon alpha that is non-glycosylated.

27. The method of claim 18 wherein the interferon is present in a pharmaceutical composition.

28. The method of claim 27 wherein the pharmaceutical composition excludes interferon other than glycosylated interferon alpha.

29. A method for treating a viral condition in a subject comprising administering to the subject a therapeutically effective amount glycosylated interferon alpha.

30. The method of claim 29 wherein the interferon alpha 2 is interferon alpha 2b.

31. The method of claim 29 wherein the viral condition is a chronic viral condition.

32. The method of claim 29 wherein the viral condition is selected from the group consisting of hepatitis B, hepatitis C, venereal warts and measles.

33. The method of claim 29 wherein the pharmaceutical composition is administered systemically to the subject.

34. The method of claim 29 wherein the viral condition is not substantially ameliorated by administering a pharmaceutical composition comprising interferon alpha that is non-glycosylated.

35. The method of claim 29 wherein the pharmaceutical composition comprises a pharmaceutical carrier.

36. The method of claim 35 wherein the pharmaceutical composition excludes interferon other than glycosylated interferon alpha 2b.

37. The method of claim 29 wherein the interferon alpha is administered in combination with at least one additional agent.

38. The method of claim 38 wherein the agent is selected from the group consisting of viramidine and ribavirin.

39. The method of claim 38 wherein the agent is administered simultaneously or sequentially.

40. The method of claim 36 wherein the viral condition is hepatitis C.

41. The method of claim 40 wherein the interferon is administered in combination with viramidine and ribavirin.
